SEQUENCE LISTING (69 sequences)

SEQ ID NO: 1 - protein, 177 aa, Homo Sapiens
  Human IL-7 (Accession number  P13232)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
  Rat IL-7 (Accession number  P56478)
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus musculus
  Mouse IL-7 (Accession number  P10168)
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Chlorocebus sabaeus
  Monkey IL-7 precursor (Accession number NP_001279008)
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
  Cow IL-7 precursor (Accession numberP26895)
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
  Sheep IL-7 precursor(Accession number  Q28540)
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 29 aa, Artificial Sequence
  GX_ori
DCDIEGKDGKQYESVLMVSIDQLLDSMKE

SEQ ID NO: 16 - protein, 32 aa, Artificial Sequence
  GX_MGM
MGMDCDIEGKDGKQYESVLMVSIDQLLDSMKE

SEQ ID NO: 17 - protein, 32 aa, Artificial Sequence
  GX_CGM
CGMDCDIEGKDGKQYESVLMVSIDQLLDSMKE

SEQ ID NO: 18 - protein, 32 aa, Artificial Sequence
  GX_AGM
AGMDCDIEGKDGKQYESVLMVSIDQLLDSMKE

SEQ ID NO: 19 - protein, 32 aa, Artificial Sequence
  GX_FGM
FGMDCDIEGKDGKQYESVLMVSIDQLLDSMKE

SEQ ID NO: 20 - protein, 32 aa, Artificial Sequence
  GX_MGA
MGADCDIEGKDGKQYESVLMVSIDQLLDSMKE

SEQ ID NO: 21 - protein, 32 aa, Artificial Sequence
  GX_MGF
MGFDCDIEGKDGKQYESVLMVSIDQLLDSMKE

SEQ ID NO: 22 - protein, 32 aa, Artificial Sequence
  GX_MGG
MGGDCDIEGKDGKQYESVLMVSIDQLLDSMKE

SEQ ID NO: 23 - protein, 32 aa, Artificial Sequence
  GX_MGS
MGSDCDIEGKDGKQYESVLMVSIDQLLDSMKE

SEQ ID NO: 24 - protein, 32 aa, Artificial Sequence
  GX_WGM
WGMDCDIEGKDGKQYESVLMVSIDQLLDSMKE

SEQ ID NO: 25 - protein, 32 aa, Artificial Sequence
  GX_GGM
GGMDCDIEGKDGKQYESVLMVSIDQLLDSMKE

SEQ ID NO: 26 - protein, 32 aa, Artificial Sequence
  GX_MGP
MGPDCDIEGKDGKQYESVLMVSIDQLLDSMKE

SEQ ID NO: 27 - protein, 32 aa, Artificial Sequence
  GX_MMM
MMMDCDIEGKDGKQYESVLMVSIDQLLDSMKE

SEQ ID NO: 28 - protein, 32 aa, Artificial Sequence
  GX_MAM
MAMDCDIEGKDGKQYESVLMVSIDQLLDSMKE

SEQ ID NO: 29 - protein, 32 aa, Artificial Sequence
  GX_MIM
MIMDCDIEGKDGKQYESVLMVSIDQLLDSMKE

SEQ ID NO: 30 - protein, 32 aa, Artificial Sequence
  GX_MTM
MTMDCDIEGKDGKQYESVLMVSIDQLLDSMKE

SEQ ID NO: 31 - protein, 32 aa, Artificial Sequence
  GX_MLM
MLMDCDIEGKDGKQYESVLMVSIDQLLDSMKE

SEQ ID NO: 32 - protein, 32 aa, Artificial Sequence
  GX_MPM
MPMDCDIEGKDGKQYESVLMVSIDQLLDSMKE

SEQ ID NO: 33 - protein, 32 aa, Artificial Sequence
  GX_MWM
MWMDCDIEGKDGKQYESVLMVSIDQLLDSMKE

SEQ ID NO: 34 - protein, 32 aa, Artificial Sequence
  GX_MCM
MCMDCDIEGKDGKQYESVLMVSIDQLLDSMKE

SEQ ID NO: 35 - protein, 32 aa, Artificial Sequence
  GX_MAV
MAVDCDIEGKDGKQYESVLMVSIDQLLDSMKE

SEQ ID NO: 36 - protein, 32 aa, Artificial Sequence
  GX_GGG
GGGDCDIEGKDGKQYESVLMVSIDQLLDSMKE

SEQ ID NO: 37 - protein, 32 aa, Artificial Sequence
  GX_IGV
IGVDCDIEGKDGKQYESVLMVSIDQLLDSMKE

SEQ ID NO: 38 - protein, 32 aa, Artificial Sequence
  GX_IAV
IAVDCDIEGKDGKQYESVLMVSIDQLLDSMKE

SEQ ID NO: 39 - protein, 32 aa, Artificial Sequence
  GX_YST
YSTDCDIEGKDGKQYESVLMVSIDQLLDSMKE

SEQ ID NO: 40 - protein, 32 aa, Artificial Sequence
  GX_LAC
LACDCDIEGKDGKQYESVLMVSIDQLLDSMKE

SEQ ID NO: 41 - protein, 32 aa, Artificial Sequence
  GX_FPW
FPWDCDIEGKDGKQYESVLMVSIDQLLDSMKE

SEQ ID NO: 42 - protein, 32 aa, Artificial Sequence
  GX_FCL
FCLDCDIEGKDGKQYESVLMVSIDQLLDSMKE

SEQ ID NO: 43 - protein, 32 aa, Artificial Sequence
  GX_RRR
RRRDCDIEGKDGKQYESVLMVSIDQLLDSMKE

SEQ ID NO: 44 - protein, 32 aa, Artificial Sequence
  GX_DDD
DDDDCDIEGKDGKQYESVLMVSIDQLLDSMKE

SEQ ID NO: 45 - protein, 32 aa, Artificial Sequence
  GX_EDQ
EDQDCDIEGKDGKQYESVLMVSIDQLLDSMKE

SEQ ID NO: 46 - protein, 32 aa, Artificial Sequence
  GX_HKR
HKRDCDIEGKDGKQYESVLMVSIDQLLDSMKE

SEQ ID NO: 47 - protein, 34 aa, Artificial Sequence
  GX_GGGGG
GGGGGDCDIEGKDGKQYESVLMVSIDQLLDSMKE

SEQ ID NO: 48 - protein, 34 aa, Artificial Sequence
  GX_MGMGM
MGMGMDCDIEGKDGKQYESVLMVSIDQLLDSMKE

SEQ ID NO: 49 - protein, 35 aa, Artificial Sequence
  GX_FPWIAV
FPWIAVDCDIEGKDGKQYESVLMVSIDQLLDSMKE

SEQ ID NO: 50 - protein, 35 aa, Artificial Sequence
  GX_FIVMAV
FIVMAVDCDIEGKDGKQYESVLMVSIDQLLDSMKE

SEQ ID NO: 51 - protein, 39 aa, Artificial Sequence
  GX_GGGGGGGGGG
GGGGGGGGGGDCDIEGKDGKQYESVLMVSIDQLLDSMKE

SEQ ID NO: 52 - protein, 39 aa, Artificial Sequence
  GX_AFCLFPWIAV
AFCLFPWIAVDCDIEGKDGKQYESVLMVSIDQLLDSMKE

SEQ ID NO: 53 - protein, 422 aa, Artificial Sequence
  IL7-hyFc (ori)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 425 aa, Artificial Sequence
  MGA IL7-hyFc
MFHVSFRYIFGLPPLILVLLPVASSMGADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 425 aa, Artificial Sequence
  MAM IL7-hyFc
MFHVSFRYIFGLPPLILVLLPVASSMAMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 425 aa, Artificial Sequence
  MGM IL7-hyFc
MFHVSFRYIFGLPPLILVLLPVASSMGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 425 aa, Artificial Sequence
  LAC IL7-hyFc
MFHVSFRYIFGLPPLILVLLPVASSLACDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 425 aa, Artificial Sequence
  IAV IL7-hyFc
MFHVSFRYIFGLPPLILVLLPVASSIAVDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 425 aa, Artificial Sequence
  RRR IL7-hyFc
MFHVSFRYIFGLPPLILVLLPVASSRRRDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - DNA, 1266 bp, Artificial Sequence
  IL7-hyFc (ori)
atgttccacgtgagcttcagatacatcttcggcctgccccccctgatcctggtgctgctgcccgtggccagcagcgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcaccaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacagaaacaccggcagaggcggcgaggagaaaaagaaggaaaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 61 - DNA, 1275 bp, Artificial Sequence
  MGA IL7-hyFc
atgttccacgtgagcttcaggtacatcttcggcctgccacccctgatcctggtgctgctgcctgtggccagctccatgggcgctgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 62 - DNA, 1275 bp, Artificial Sequence
  MAM IL7-hyFc
atgttccacgtgagcttcaggtacatcttcggcctgccacccctgatcctggtgctgctgcctgtggccagctccatggctatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 63 - DNA, 1275 bp, Artificial Sequence
  MGM IL7-hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 64 - DNA, 1275 bp, Artificial Sequence
  LAC IL7-hyFc
atgttccacgtgagcttcaggtacatcttcggcctgccacccctgatcctggtgctgctgcctgtggccagctccctggcctgcgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 65 - DNA, 1275 bp, Artificial Sequence
  IAV IL7-hyFc
atgttccacgtgagcttcaggtacatcttcggcctgccacccctgatcctggtgctgctgcctgtggccagctccatcgccgtggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 66 - DNA, 1275 bp, Artificial Sequence
  RRR IL7-hyFc
atgttccacgtgagcttcaggtacatcttcggcctgccacccctgatcctggtgctgctgcctgtggccagctcccgtaggcgcgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 67 - protein, 10 aa, Artificial Sequence
  N-terminal sequence of wild-type IL-7
DCDIEGKDGK

SEQ ID NO: 68 - protein, 6 aa, Artificial Sequence
  N-terminal sequence of wild-type IL-7
DCDIEG

SEQ ID NO: 69 - protein, 9 aa, Artificial Sequence
  N-terminal sequence of modified IL-7
MGMDCDIEG

(19) United States
(12) Patent Application Publication
(10) Pub. No.: US 2021/0324027 A1
(43) Pub. Date: Oct. 21, 2021

(54) MODIFIED INTERLEUKIN-7 PROTEINS AND USES THEREOF

(71) Applicants: GENEXINE, INC., Seongnam-si (KR); NEOIMMUNE TECH, INC., Rockville, MD (US)

(72) Inventors: Young Chul SUNG, Seoul (KR); Gwang Hyeon GWON, Seongnam-si (KR); So Young CHOI, Yongin-si (KR); Hyi Jeong JI, Seoul (KR); Dong Hoon CHOI, Seongnam-si (KR)

(73) Assignees: GENEXINE, INC., Seongnam-si, (KR); NEOIMMUNE TECH, INC., Rockville, MD (US)

(21) Appl. No.: 17/232,790

(22) Filed: Apr. 16, 2021

(30) Foreign Application Priority Data
Apr. 16, 2020 (KR) 10-2020-0045707
Apr. 16, 2020 (KR) 10-2020-0045708
Apr. 16, 2020 (KR) 10-2020-0045709

(51) Int. Cl. C07K 14/54

(52) U.S. Cl. CPC C07K 14/5418; C07K 2319/30; C07K 2319/31

(57) ABSTRACT

Provided are a modified IL-7 polypeptide and a fusion protein containing the modified IL-7 polypeptide. The fusion protein of the modified IL-7 includes: a first domain containing an interleukin-7 polypeptide; a second domain containing an oligopeptide having 1 to 10 amino acid residues (with proviso that the second domain excludes the oligopeptide consisting of methionine and/or glycine); and (c) a third domain which prolongs the half-life of the IL-7 fusion protein. The modified IL-7 polypeptide is composed of the (a) first domain and the (b) second domain. The modified IL-7 polypeptide and the fusion protein are expressed in a higher yield than the wild-type IL-7 and shows increased stability.

### CROSS REFERENCE TO RELATED APPLICATIONS

This application claims priority and benefit under 35 U.S.C. § 119 based on Korean Patent Application Nos. KR 10-2020-0045707, KR 10-2020-0045708, and KR 10-2020-0045709, each filed on Apr. 16, 2020, the contents of which are incorporated herein by reference in their entireties.

### SEQUENCE LISTING

The content of the electronically submitted sequence listing, file name: Sequence_Listing_As_Filed.txt; size: 79,510 bytes; and date of creation: Apr. 11, 2021, filed herewith, is incorporated herein by reference in its entirety.

### TECHNICAL FIELD

The disclosure relates to modified interleukin-7 protein with improved expression compared to corresponding wild-type interleukin-7 and fusion proteins comprising the modified interleukin-7 polypeptide and a domain extending the half-life of the interleukin-7 and uses thereof.

### BACKGROUND ART

Interleukin-7 (IL-7) is a cytokine stimulating immune responses mediated by T cell and B cell, and specifically, it plays an important role in an adaptive immune system. Although IL-7 is mainly secreted by stromal cells in the bone marrow and the thymus, it is also produced in keratinocytes, dendritic cells, hepatocytes, neural and epithelial cells.

IL-7 actives the immune system by influencing the survival and differentiation of T cell and B cell, and stimulating the activity of NK (natural killer) cell, and so on, and specifically, it plays an important role in the development of B cell. IL-7 binds to HGF (hepatocyte growth factor) to function as a pre-pro-B growth-stimulating factor, V(D)J rearrangement cofactor of T cell receptor beta (TCRβ). And, it has been reported based on clinical trial studies that IL-7 modulates development of lymph nodes through LTi (lymphoid tissue inducer) cells, and promotes expansion and survival of naive T cells and/or memory T cells. IL-7 enhances the immune response in the human body by stimulating the secretion of IL-2, a type of cytokine, and interferon-γ.

Due to its biological activities of promoting survival and proliferation of T-cells, B-cells and other immune cells, IL-7 is regarded as an excellent candidate for an immune therapeutic agent applicable to various disorders such as viral infections, cancer, and immune system damages.

Cancer cells form immune-suppressive environments and evade the host immune surveillance to continue proliferation. Standard cancer treatments such as chemotherapy or radiation therapy cause a significant immunosuppression where the number of T cells reduces and cytotoxic T lymphocytes, effector T cells and macrophages, which are infiltrated into cancer tissues, fail to stop cancer cells proliferation.

An immunotherapy using an immune modulator has gained spotlight, and several clinical trials employing an immune modulator in combination with chemotherapy or radiation therapy are carried out. IL-7 has received an attention for its effectiveness in reversing lymphopenia (i.e., decreased number of T cells) in patients undergoing chemotherapy or radiation therapy and in enhancing immune system in the patients. IL-7 is expected to be useful in inhibiting or treating T cell exhaustions in chronic infections including virus infections such as HIV, HBV, HCV, SIV, and the like.

It is well known that wild type IL-7 has very short life span in living body. It is also known that IL-7 is very difficult to produce for commercial uses due to its low yields. Attempts have been made to increase a production yield by denaturation and refolding of IL-7 to produce IL-7 conformers. For example, U.S. Pat. No. 7,585,947, of which the content is incorporated herein by reference. The process described in U.S. Pat. No. 7,585,947 has drawbacks that it requires complex denaturation steps.

Therefore, there have been needs for a modified IL-7 or variant IL-7 which enables an increased production yield using a relatively simple process. The inventors previously developed modified IL-7 polypeptides, as described in U.S. patent application Ser. No. 15/126,313, of which the entire content is incorporated herein by reference. Various uses of the modified IL-7 and/or its fusion proteins are disclosed in, for example, PCT/US2020/049483 and PCT/US2020/049483, the entire contents of which are incorporated herein by reference.

### SUMMARY

An aspect of the present disclosure provides a modified IL-7.

Another aspect of the present disclosure provide a fusion protein comprising the modified IL-7.

An aspect of the present disclosure provides a nucleic acid encoding the modified IL-7 or the fusion proteins, a vector including the nucleic acid and a host cell including the vector, and a method for preparing a modified IL-7 or its fusion proteins including the same.

Another aspect of the present disclosure provides a pharmaceutical composition comprising the modified IL-7 or its fusion proteins, and uses thereof.

Still another aspect of the present disclosure provides methods of utilizing the modified IL-7 or fusion proteins in treating or improving lymphopenia in a subject in need thereof. In an embodiment, the subject in need of treating or improving lymphopenia is suffering from a cancer; infection; chronic failure of the right ventricle of the heart; Hodgkin's disease; a leak or rupture in the thoracic duct; side effects of prescription medications including anticancer agents (e.g., chemotherapy), antiviral agents, or glucocorticoids; malnutrition resulting from diets that are low in protein, radiation therapy, uremia, autoimmune disorders, immune deficiency syndromes, thymectomy, or a combination thereof; or idiopathic, acute radiation syndrome (ARS) or a combination thereof. In an embodiment, the subject may be human.

Still another aspect of the present disclosure provides methods of utilizing the modified IL-7 or its fusion proteins in treating cancer in a subject in need thereof. The subject in need thereof may be a mammal suffering from a cancer or a malignant tumor. The cancer may be a solid tumor. In an exemplary embodiment, the solid tumor may be locally advanced or metastatic solid tumors or glioblastoma. In an embodiment, the subject may be human. In an embodiment, the subject may previously or concurrently undergo a cancer treatment. The modified IL-7 or its fusion protein may be administered separately or simultaneously with another anti-cancer drug(s).

An aspect of the disclosure provides the use of the modified IL-7 or its fusion proteins in manufacturing medicaments for treating cancer or for improving or treating lymphopenia described above.

In aspects of the present disclosure, the modified IL-7 is represented by the following formula (I):

A-IL-7 formula (I)
wherein A is an oligopeptide consisting of 1 to 50 amino acid residues, and
the IL-7 is a polypeptide which is capable of binding to IL-7 receptor,
with proviso that the A excludes an oligopeptide consisting of methionine and/or glycine.

In aspects of the present disclosure, the IL-7 has an amino acid sequence selected from the group consisting of SEQ ID NOS: 1 to 6. In other aspects, the IL-7 is a polypeptide having about 55% or more, about 60% or more, about 65% or more, about 70% or more, about 75% or more, about 80% or more, about 85% or more, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more, about 99% or more of sequence identity to SEQ ID NO: 1.

In an aspect, the A may be linked to the N-terminal of the IL-7.

In an aspect, the A may be an oligopeptide consisting of 1 to 10 amino acid residues, and wherein the modified interleukin-7 has an increased stable helix structure than the counterpart IL-7 without A.

In an aspect, A may be an oligopeptide selected from the group consisting of (a)-(c):
(a) an oligopeptide comprising methionine and/or glycine, and one or more hydrophobic amino acid residues selected from the group consisting of isoleucine, valine, leucine, phenylalanine, cysteine, alanine, tryptophan, and proline,
(b) an oligopeptide consisting of a hydrophobic amino acid residue selected from the group consisting of isoleucine, valine, leucine, phenylalanine, cysteine, alanine, tryptophan and proline, and
(c) an oligopeptide consisting of a hydrophilic amino acid residue selected from the group consisting of asparagine, aspartic acid, glutamine, glutamic acid, lysine, arginine, histidine, tyrosine, threonine and serine.

In another aspect, the A is any one selected from the group consisting of alanine, cysteine, methionine-alanine, methionine-alanine-methionine, methionine-alanine-methionine-methionine-alanine-methionine, methionine-alanine-methionine-methionine-alanine-methionine-methionine-alanine-methionine, cysteine-glycine-methionine, cysteine-glycine-methionine-methionine-alanine-methionine, cysteine-glycine-methionine-cysteine-glycine-methionine, alanine-glycine-methionine, cysteine-glycine-methionine-alanine-glycine-methionine, methionine-isoleucine-methionine, methionine-alanine-methionine-isoleucine-methionine-methionine-isoleucine-methionine-methionine-methionine, cysteine-glycine-methionine-alanine-glycine-methionine, methinone-alanine-methionine-alanine-methionine-glycine-methionine-methionine-glycine-glycine, arginine-arginine-arginine, methionine-threonine-methionine, phenylalanine-glycine-methionine, methionine-leucine-methionine, methionine-glycine-alanine, methionine-alanine-valine, methionine-glycine-phenylalanine, aspartic acid-aspartic acid-aspartic acid, methionine-proline-methionine, methionine-glycine-serine, isoleucine-glycine-valine, methionine-tryptophan-methionine, glutamic acid-aspartic acid-glutamine, isoleucine-alanine-valine, methionine-cysteine-methionine, alanine-phenylalanine-cysteine-leucine-phenylanaline-proline-tryptophan-isoleucine-alanine-valine, tyrosine-serine-threonine, histidine-lysine-arginine, tryptophan-glycine-methionine, methionine-glycine-proline, leucine-alanine-cysteine, phenylalanine-proline-tryptophan, phenylalanine-cysteine-leucine, phenylalanine-proline-tryptophan-isoleucine-alanine-valine, phenylalanine-isoleucine-valine-methionine-alanine-valine.

In still another aspect, the A is an oligopeptide comprising 2-10 amino acid residues, and the oligopeptide comprises
(i) methionine and alanine;
(ii) methionine and cysteine;
(iii) methionine and valine;
(iv) methionine and phenylalanine;
(v) methionine and leucine;
(vi) methionine and serine;
(vii) methionine and threonine;
(viii) methionine and tryptophan;
(ix) methionine and proline;
(x) methionine and isoleucine;
(xi) methionine, glycine, and alanine;
(xii) methionine, glycine, and cysteine;
(xiii) methionine, glycine, and valine;
(xiv) methionine, glycine, and phenylalanine;
(xv) methionine, glycine, and leucine;
(xvi) methionine, glycine, and serine;
(xvii) methionine, glycine, and threonine;
(xviii) methionine, glycine, and tryptophan;
(xix) methionine, glycine, and isoleucine; or
(xx) methionine, glycine, and proline.

In another aspect, the fusion protein of the modified IL-7 comprises: a first domain comprising an interleukin-7, (b) a second domain comprising an oligopeptide having 1 to 50 amino acid residues (with proviso that the second domain excludes the oligopeptide consisting of methionine and/or glycine); and (c) a third domain which prolongs the half-life of the IL-7 fusion protein.

In embodiments of the fusion protein, the third domain may be linked to the N-terminal or C-terminal of the first domain or the second domain. In an embodiment, the third domain may be linked to the C-terminal of the second domain.

In embodiments of the fusion protein, the third domain may be any one selected from the group consisting of an Fc region of immunoglobulin or a part thereof, albumin, an albumin-binding polypeptide, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof. In an embodiment, the third domain comprises an Fc region of a modified immunoglobulin. In an embodiment, the modified immunoglobulin is selected from the group consisting of IgG1, IgG2, IgG3, IgG4, IgA1, IgA2, IgD, IgE and a combination thereof.

In embodiments of the fusion protein, the Fc region of the modified immunoglobulin comprises a hinge region, a CH2 domain, and a CH3 domain from the N-terminal to the C-terminal direction, wherein the hinge region comprises a human IgD hinge region, the CH2 domain comprises a part of the amino acid residues of CH2 domain of human IgD and human IgG4, and the CH3 domain comprises a part of the amino acid residues of the human IgG4 CH3 domain.

In embodiments of the fusion protein, the Fc region of the modified immunoglobulin may be represented by the following Formula (II):

N′—(Z1)p-Y—Z2-Z3-Z4-C′ Formula (II)

wherein N′ is the N-terminal of a polypeptide and C′ is the C-terminal of a polypeptide;

p is an integer of 0 or 1;

Z1 is an amino acid sequence having 5 to 9 consecutive amino acid residues starting from the amino acid residue at position 98 of the positions from 90 to 98 of SEQ ID NO: 7;

Y is an amino acid sequence having 5 to 64 consecutive amino acid residues starting from the amino acid residue at position 162 of the positions from 99 to 162 of SEQ ID NO: 7;

Z2 is an amino acid sequence having 4 to 37 consecutive amino acid residues starting from the amino acid residue at position 163 of the positions from 163 to 199 of SEQ ID NO: 7;

Z3 is an amino acid sequence having 71 to 106 consecutive amino acid residues starting from the amino acid residue at position 220 of the positions from 115 to 220 of SEQ ID NO: 8; and

Z4 is an amino acid sequence having 80 to 107 consecutive amino acid residues starting from the amino acid residue at position 221 of the positions from 221 to 327 of SEQ ID NO: 8.

According to one embodiments, the third domain has an amino acid sequence selected from the group consisting of SEQ ID NOS: 9 to 14.

### BRIEF DESCRIPTION OF DRAWINGS

FIG. 1 is a schematic diagram showing the structure of a modified IL-7 in which the N-terminal comprises the sequence of DCDIEGKGK (SEQ ID NO: 67) in which the first six amino acid residues DCDIEG (SEQ ID NO: 68) is disordered.

FIG. 2 illustrates a model of stabilized structure of modified IL-7 by introducing an oligopeptide at the N-terminal of the IL-7.

FIG. 3A and FIG. 3B show the amino acid sequence of various modified IL-7 polypeptides with their names as used herein and the number of amino acid residues thereof.

FIG. 4A and FIG. 4B show analysis results of the various modified IL-7 polypeptide CD according to certain embodiments.

FIG. 5 shows analysis results of some of the modified IL-7 polypeptide CD according to certain embodiments, depending on the surrounding environment.

FIG. 6 shows protein expression levels of wild type IL-7 fusion protein and modified IL-7 fusion proteins according to embodiments, in which the IL-7 polypeptide is fused to an Fc domain, and SDS-PAGE analysis to compare the level of expressions of a fusion protein comprising a wild-type IL-7 and Fc region (“Ori”) and various modified IL-7 fusion proteins.

FIG. 7A and FIG. 7B summarize the results of various modified IL-7 polypeptide CD and the protein expression levels of fusion proteins of various modified IL-7 polypeptide.

FIG. 8 shows protein expression levels of wild type IL-7 fusion protein and modified IL-7 fusion proteins according to embodiments, in which the IL-7 polypeptide is fused to an Fc domain, and SDS-PAGE analysis to compare the level of expressions of a fusion protein comprising a wild-type IL-7 and Fc region (“Ori”) and various modified IL-7 fusion proteins.

FIG. 9A and FIG. 9B summarize the results of various modified IL-7 polypeptide CD and the protein expression levels of fusion proteins of various modified IL-7 polypeptide.

### EMBODIMENTS

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the aspects provided herein, because the scope of the aspects provided herein is limited only by the claims. Unless otherwise defined, all technical and scientific terms used herein have the same meaning as commonly understood by one of ordinary skill in the art to which aspects provided herein belong.

The term “pharmaceutical composition” is defined herein to refer to a mixture or solution containing at least one therapeutic agent to be administered to a mammal, in order to prevent or treat a particular disease or condition affecting the mammal. In embodiments, the mammal may be human.

The term “pharmaceutically acceptable” is defined herein to refer to those compounds, materials, compositions and/or dosage forms, which are, within the scope of sound medical judgment, suitable for contact with the tissues a human patient without excessive toxicity, irritation allergic response and other problem complications commensurate with a reasonable benefit/risk ratio.

The term “treating” or “treatment” as used herein comprises a treatment relieving, decrease, reducing or alleviating at least one symptom in a human patient or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent delay or treat, or all, as appropriate, development or continuance or aggravation of a disease in a subject.

The term “prevent”, “preventing” or “prevention” as used herein comprises the prevention of at least one symptom associated with or caused by the state, disease or disorder being prevented.

The term “pharmaceutically effective amount” or “clinically effective amount” of a combination of therapeutic agents is an amount sufficient to provide an observable improvement over the baseline clinically observable signs and symptoms of the disorder treated with the combination.

The phrase “a human patient in need of such treatment” as used herein refers to a human patient diagnosed with or suffering from the identified proliferative disease.

The term about” or “approximately” shall have the meaning of within 10%, 9%, 8%, 7%, 6%, or within 5%, of a given value or range.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

### Modified IL-7

A modified IL-7 that can be used in the embodiments may have the following structure:

A-IL-7 formula (I)

wherein A is an oligopeptide consisting of 1 to 50 amino acid residues (with proviso that A excludes an oligopeptide consisting of glycine and/or methionine), and IL-7 is an interleukin 7, a polypeptide capable of binding to IL-7 receptor (also known as CD127), or a polypeptide having the activity of IL-7 or a similar activity thereto.

As used herein, the term “a polypeptide having the activity of IL-7 or a similar activity thereof” refers to a polypeptide or protein having the same or similar sequence and activity to IL-7. Unless otherwise specified in an embodiment, the term can be used as a concept which is interchangeable with the first domain of the IL-7 fusion protein or the modified IL-7 fusion protein, as interchangeably used herein.

The IL-7 includes a polypeptide comprising the amino acid sequence of SEQ ID NO: 1, 2, 3, 4, 5, or 6. Additionally, the IL-7 may have a sequence identity of about 55% or higher, about 60% or higher, about 65% or higher, about 70% or higher, about 75% or higher, about 80% or higher, about 85% or higher, about 86% or higher, about 87% or higher, about 88% or higher, about 89% or higher, about 90% or higher, about 91% or higher, about 92% or higher, about 93% or higher, about 94% or higher, about 95% or higher, about 96% or higher, about 97% or higher, about 98%, or about 99% or higher to the sequence of SEQ ID NO: 1, 2, 3, 4, 5, or 6. The sequence identity of the peptide sequence may be determined by using a known sequence alignment or comparison software. For example, the sequence identity may be determined by using the BLASTP program (blast.ncbi.nlm.nih.gov/Blast.cgi?PAGE=Proteins) with its default setting.

The IL-7 may include an IL-7 protein or a fragment thereof, wherein the fragment is capable of binding to IL-7 receptor. As used herein, the term “IL-7 protein” may be used as a concept to include “IL-7 protein and a fragment thereof, wherein the fragment is capable of binding to IL-7 receptor.” And, IL-7 includes a secreted form, i.e., a peptide from which the signal sequence is removed.

The IL-7 may be one obtained from humans, rats, mice, monkeys, cows, or sheep.

Unless otherwise specified, the terms “protein”, “polypeptide”, and “peptide” may be used as an interchangeable concept.

Specifically, human IL-7 may have the amino acid sequence of SEQ ID NO: 1 (Genbank Accession No. P13232); rat IL-7 may have the amino acid sequence represented by SEQ ID NO: 2 (Genbank Accession No. P56478); mouse IL-7 may have the amino acid sequence represented by SEQ ID NO: 3 (Genbank Accession No. P10168); monkey IL-7 may have the amino acid sequence represented by SEQ ID NO: 4 (Genbank Accession No. NP_001279008); cow IL-7 may have the amino acid sequence represented by SEQ ID NO: 5 (Genbank Accession No. P26895), and sheep IL-7 may have the amino acid sequence represented by SEQ ID NO: 6 (Genbank Accession No. Q28540).

Additionally, the IL-7 protein or a fragment thereof may include variously modified proteins or peptides, i.e., variants. The above modification may be performed by a method of a substitution, a deletion, or an addition of at least one protein to the wild type IL-7, without modifying the function of the IL-7. These various proteins or peptides may have a sequence identify of at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the wild type protein.

Conventionally, a wild type amino acid residue is substituted with alanine, but the substitution may be performed a conservative amino acid substitution, which does not affect or gives a weak effect on the entire protein charge, i.e., polarity or hydrophobicity.

For the conservative amino acid substitution, Table 1 below may be referred to.

TABLE 1

| | |
|---|---|
| Basic | Arginine (Arg, R) |
| | Lysine (Lys, K) |
| | Histidine (His, H) |
| Acidic | Glutamic acid (Glu, E) |
| | Aspartic acid (Asp, D) |
| Uncharged polar | Glutamine (Gln, O) |
| | Asparagine (Asn, N) |
| | Serine (Ser, S) |
| | Threonine (Thr, T) |
| | Tyrosine (Tyr, Y) |
| Non-polar | Phenylalanine (Phe, F) |
| | Tryptophan (Trp, W) |
| | Cystein (Cys, C) |
| | Glycine (Gly, G) |
| | Alanine (Ala, A) |
| | Valine (Val, V) |
| | Proline (Pro, P) |
| | Methionine (Met, M) |
| | Leucine (Leu, L) |
| | Norleucine |
| | Isoleucine |

For each amino acid, additional conservative substitution includes “a homolog” of the amino acid. In particular, the “homolog” refers to an amino acid, in which a methylene group (CH2) is inserted to the side chain of the beta position of the side chain of the amino acid. Examples of the “homolog” may include homophenylalanine, homoarginine, homoserine, etc., but is not limited thereto.

In the structure of the modified IL-7 of A-IL-7, the moiety A may be directly linked to the N-terminal of IL-7, or linked through a linker, and unless otherwise specified, the term may be used as a concept which can be interchangeable with the second domain of IL-7 fusion proteins.

In an embodiment, A may be linked to the N-terminal of IL-7. The A is characterized in that it includes 1 to 50 amino acid residues, and the amino acid may be selected from any amino acid residues, with proviso that the A excludes an oligopeptide consisting of methionine and/or glycine. In an embodiment, A may be an oligopeptide comprising 1 to 30 amino acid residues, 1 to 25 amino acid residues, 1-20 amino acid residues, 1-15 amino acid residues, 1-10 amino acid residues, 1-9 amino acid residues, 1-8 amino acid residues, 1-7 amino acid residues, 1-6 amino acid residues, 1-5 amino acid residues, 2 to 30 amino acid residues, 2 to 25 amino acid residues, 2-20 amino acid residues, 2-15 amino acid residues, 2-10 amino acid residues, 2-9 amino acid residues, 2-8 amino acid residues, 2-7 amino acid residues, 2-6 amino acid residues, or 2-5 amino acid residues, with proviso that the A excludes an oligopeptide consisting of methionine and/or glycine.

In an embodiment, A may be an oligopeptide selected from the group consisting of (a)-(c):

(a) an oligopeptide comprising methionine and/or glycine, and one or more hydrophobic amino acid residues selected from the group consisting of isoleucine, valine, leucine, phenylalanine, cysteine, alanine, tryptophan, and proline,

(b) an oligopeptide consisting of a hydrophobic amino acid residue selected from the group consisting of isoleucine, valine, leucine, phenylalanine, cysteine, alanine, tryptophan, and proline, and

(c) an oligopeptide consisting of a hydrophilic amino acid residue selected from the group consisting of asparagine, aspartic acid, glutamine, glutamic acid, lysine, arginine, and histidine, tyrosine, threonine, and serine.

In another embodiment, the A may be an oligopeptide comprising amino acid residues listed in (i)-(xx):
(i) methionine and alanine;
(ii) methionine and cysteine;
(iii) methionine and valine;
(iv) methionine and phenylalanine;
(v) methionine and leucine;
(vi) methionine and serine;
(vii) methionine and threonine;
(viii) Methionine and tryptophan;
(ix) methionine and proline;
(x) methionine and isoleucine;
(xi) methionine, glycine, and alanine;
(xii) methionine, glycine, and cysteine;
(xiii) methionine, glycine, and valine;
(xiv) methionine, glycine, and phenylalanine;
(xv) methionine, glycine, and leucine;
(xvi) methionine, glycine, and serine;
(xvii) methionine, glycine, and threonine;
(xviii) methionine, glycine, and tryptophan;
(xix) methionine, glycine, and isoleucine; or
(xx) methionine, glycine, and proline.

In an embodiment, A may be any one selected from the group consisting of alanine, cysteine, methionine-alanine, methionine-alanine-methionine, methionine-alanine-methionine-methionine-alanine-methionine, methionine-alanine-methionine-methionine-alanine-methionine-methionine-alanine-methionine, cysteine-glycine-methionine, cysteine-glycine-methionine-methionine-alanine-methionine, cysteine-glycine-methionine-cysteine-glycine-methionine, alanine-glycine-methionine, cysteine-glycine-methionine-alanine-glycine-methionine, methionine-isoleucine-methionine, methionine-threonine-methionine, methionine-alanine-methionine-isoleucine-methionine-methionine-isoleucine-methionine-methionine-methionine, cysteine-glycine-methionine-alanine-glycine-methionine, methinone-alanine-methionine-alanine-methionine-glycine-methionine-methionine-glycine-glycine, arginine-arginine-arginine, methionine-threonine-methionine, phenylalanine-glycine-methionine, methionine-leucine-methionine, methionine-glycine-alanine, methionine-alanine-valine, methionine-glycine-phenylalanine, aspartic acid-aspartic acid-aspartic acid, aspartic acid-aspartic acid-aspartic acid-aspartic acid, methionine-proline-methionine, methionine-glycine-serine, isoleucine-glycine-valine, methionine-tryptophan-methionine, glutamic acid-aspartic acid-glutamine, isoleucine-alanine-valine, methionine-cysteine-methionine, alanine-phenylalanine-cysteine-leucine-phenylanaline-proline-tryptophan-isoleucine-alanine-valine, tyrosine-serine-threonine, histidine-lysine-arginine, tryptophan-glycine-methionine, methionine-glycine-proline, leucine-alanine-cysteine, phenylalanine-proline-tryptophan, phenylalanine-cysteine-leucine, phenylalanine-proline-tryptophan-isoleucine-alanine-valine, phenylalanine-isoleucine-valine-methionine-alanine-valine.

In an embodiment, the modified IL-7 may comprise any one of the sequence selected from the group consisting of SEQ ID NOS: 17-21, 23, 24, 26, 28-35, 37-46, 49, 50, and 52.

### Fusion Protein of Modified IL-7 or Modified IL-7 Fusion Protein

Another aspect provides an IL-7 fusion protein, comprising: a first domain comprising a polypeptide having the activity of IL-7 or a similar activity thereof; a second domain including an amino acid sequence having 1 to 10 amino acid residues consisting of methionine, glycine, or a combination thereof; and a third domain which prolongs the half-life of the IL-7 fusion protein.

The third domain may be linked to the N-terminal or the C-terminal of the first domain or the second domain. Additionally, the modified IL-7 including the first domain and the second domain may be linked to both terminals of the third domain.

The third domain may be a fusion partner for increasing in vivo half-life, and preferably, may include any one selected from the group consisting of an Fc region of immunoglobulin or a part thereof, albumin, an albumin-binding polypeptide, Pro/Ala/Ser (PAS), C-terminal peptide (CTP) of 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof.

When the third domain is an Fc region of immunoglobulin it may be an Fc region of a modified immunoglobulin. In particular, the Fc region of the modified immunoglobulin may be one in which the antibody-dependent cellular cytotoxicity (ADCC) or complement-dependent cytotoxicity (CDC) weakened due to the modification in the binding affinity with the Fc receptor and/or a complement. The modified immunoglobulin may be selected from the group consisting of IgG1, IgG2, IgG3, IgG4, IgA1, IgA2, IgD, IgE and a combination thereof. Specifically, the Fc region of the modified immunoglobulin may include a hinge region, a CH2 domain, and a CH3 domain from the N-terminal to the C-terminal. In particular, the hinge region may include the human IgD hinge region; the CH2 domain may include a part of the amino acid residues of the human IgD and a part of the amino acid residues of the human IgG4 CH2 domain; and the CH3 domain may include a part of the amino acid residues of the human IgG4 CH3 domain.

Additionally, two fusion proteins may form a dimer, for example, when the third domain is an Fc region, the Fc regions may bind to each other and thereby form a dimer.

As used herein, the terms “Fe region”, “Fe fragment”, or “Fe” refers to a protein which includes the heavy chain constant region 2 (CH2) and the heavy chain constant region 3 (CH3) of immunoglobulin but does not include its variable regions of the heavy chain and the light chain and the light chain constant region (CL1), and it may further include a hinge region of the heavy chain constant region. In an embodiment, a hybrid Fc or a hybrid Fc fragment thereof may be called “hFc” or “hyFc.”

Additionally, as used herein, the term “an Fc region variant” refers to one which was prepared by substituting a part of the amino acids among the Fc region or by combining the Fc regions of different kinds. The Fc region variant can prevent from being cut off at the hinge region. Specifically, the 144th amino acid and/or 145th amino acid of SEQ ID NO: 9 may be modified. Preferably, the variant may be one, in which the 144th amino acid, K, was substituted with G or S, and one, in which the 145th amino acid, E, was substituted with G or S.

Additionally, the Fc region or the Fc region variant of the modified immunoglobulin may be represented by the following Formula (I):

N′—(Z1)p-Y—Z2-Z3-Z4-C′. Formula (I)

In the above Formula (I),
N′ is the N-terminal of a polypeptide and C′ is the C-terminal of a polypeptide;
p is an integer of 0 or 1;
Z1 is an amino acid sequence having 5 to 9 consecutive amino acid residues from the amino acid residue at position 98 toward the N-terminal, among the amino acid residues at positions from 90 to 98 of SEQ ID NO: 7;
Y is an amino acid sequence having 5 to 64 consecutive amino acid residues from the amino acid residue at position 162 toward the N-terminal, among the amino acid residues at positions from 99 to 162 of SEQ ID NO: 7;
Z2 is an amino acid sequence having 4 to 37 consecutive amino acid residues from the amino acid residue at position 163 toward the C-terminal, among the amino acid residues at positions from 163 to 199 of SEQ ID NO: 7;
Z3 is an amino acid sequence having 71 to 106 consecutive amino acid residues from the amino acid residue at position 220 toward the N-terminal, among the amino acid residues at positions from 115 to 220 of SEQ ID NO: 8; and
Z4 is an amino acid sequence having 80 to 107 consecutive amino acid residues from the amino acid residue at position 221 toward the C-terminal, among the amino acid residues at positions from 221 to 327 of SEQ ID NO: 8.

Additionally, the Fc fragment may be in the form of having native sugar chains, increased sugar chains, or decreased sugar chains compared to the native form, or may be in a deglycosylated form. The immunoglobulin Fc sugar chains may be modified by conventional methods such as a chemical method, an enzymatic method, and a genetic engineering method using a microorganism. The removal of sugar chains from an Fc fragment results in a sham decrease in binding affinity to the Clq part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of an embodiment as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

Additionally, the Fc region of the modified immunoglobulin may include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). Additionally, the Fc region of the modified immunoglobulin may include the amino acid sequence of SEQ ID NO: 14 (a non-lytic mouse Fc).

The Fc region of the modified immunoglobulin may be one described in U.S. Pat. No. 7,867,491, and the production of the Fc region of the modified immunoglobulin may be performed referring to the disclosure in U.S. Pat. No. 7,867,491, the entire content of which is incorporated herein by reference.

The second domain may be directly linked to the N-terminal of the first domain or linked by a linker. Specifically, the result may be in the form of the second domain-the first domain or the second domain-linker-the first domain.

The third domain may be directly linked to the first domain or the second domain or linked by a linker. Specifically, the result may be in the form of the second domain-the first domain-the third domain, the third domain-the second domain-the first domain, the second domain-the first domain-linker-the third domain, the third domain-linker-the second domain-the first domain, the second domain-linker-the first domain-linker-the third domain, or the third domain-linker-the second domain-the first domain.

When the linker is a peptide linker, the connection may occur in any linking region. They may be coupled using a crosslinking agent known in the art. Examples of the crosslinking agent may include N-hydroxysuccinimide esters such as 1,1-bis(diazoacetyl)-2-phenylethane, glutaraldehyde, and 4-azidosalicylic acid; imidoesters including disuccinimidyl esters such as 3,3′-dithiobis(succinimidylpropionate), and bifunctional maleimides such as bis-N-maleimido-1,8-octane, but is not limited thereto.

Additionally, the linker may be an albumin linker or a peptide linker. The peptide linker may be a peptide of 10 to 20 amino acid residues consisting of Gly and Ser residues.

When the linker is formed by one selected from the group consisting of a chemical bond, the chemical bond may be a disulfide bond, a diamine bond, a sulfide-amine bond, a carboxy-amine bond, an ester bond, and a covalent bond.

The modified IL-7 may have a structure of A-IL-7 including a polypeptide having the activity of IL-7 or a similar activity thereof and an oligopeptide consisting of 1 to 10 amino acids.

In an embodiment, the modified IL-7 may have an amino acid sequence selected from the group consisting of SEQ ID NOS: 15 to 20. Additionally, the modified IL-7 may have a sequence having a sequence identity of at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the amino acid sequence of SEQ ID NOS: 15, 16, 17, 18, 19, or 20.

In another exemplary embodiment, the modified IL-7 or an IL-7 fusion protein, which comprising a first domain including a polypeptide having the activity of IL-7 or a similar activity thereof; a second domain comprising an amino acid sequence having 1 to 10 amino acid residues consisting of methionine, glycine, or a combination thereof; and a third domain, which is an Fc region of modified immunoglobulin, coupled to the C-terminal of the first domain.

The IL-7 fusion protein may have an amino acid sequence selected from the group consisting of SEQ ID NOS: 21 to 25. Additionally, the IL-7 fusion protein may have a sequence having a sequence identity of at least about 70%, at least about 75%, at least about 80%, 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the amino acid sequence of SEQ ID NOS: 21, 22, 23, 24, or 25.

The IL-7 fusion protein of SEQ ID NOS: 21 to 25 may be encoded by a polynucleotide sequence selected from the group consisting of SEQ ID NOS: 29 to 39.

The nucleic acid molecule may further include a signal sequence or a leader sequence.

As used herein, the term “signal sequence” refers to a fragment directing the secretion of a biologically active molecule drug and a fusion protein, and it is cut off after being translated in a host cell. The signal sequence of an embodiment is a polynucleotide encoding an amino acid sequence initiating the movement of the protein penetrating the endoplasmic reticulum (ER) membrane. The useful signal sequences in an embodiment include an antibody light chain signal sequence, e.g., antibody 14.18 (Gillies et al., J. Immunol. Meth 1989. 125:191-202), an antibody heavy chain signal sequence, e.g., MOPC141 an antibody heavy chain signal sequence (Sakano et al., Nature, 1980. 286: 676-683), and other signal sequences know in the art (e.g., see Watson et al., Nucleic Acid Research, 1984. 12:5145-5164).

The characteristics of the signal peptides are well known in the art, and the signal peptides conventionally having 16 to 30 amino acids, but they may include more or less number of amino acid residues. Conventional signal peptides consist of three regions of the basic N-terminal region, a central hydrophobic region, and a more polar C-terminal region.

The central hydrophobic region includes 4 to 12 hydrophobic residues, which immobilize the signal sequence through a membrane lipid bilayer during the translocation of an immature polypeptide. After the initiation, the signal sequence is frequently cut off within the lumen of ER by a cellular enzyme known as a signal peptidase. In particular, the signal sequence may be a secretory signal sequence for tissue plasminogen activation (tPa), signal sequence of herpes simplex virus glycoprotein D (HSV gDs), or a growth hormone. Preferably, the secretory signal sequence used in higher eukaryotic cells including mammals, etc., may be used. Additionally, as the secretory signal sequence, the signal sequence included in the wild type IL-7 may be used or it may be used after substituting with a codon with high expression frequency in a host cell.

An isolated nucleic acid molecule encoding the modified IL-7 or an IL-7 fusion protein may be contained in an expression vector.

As used herein, the term “vector” is understood as a nucleic acid means which includes a nucleotide sequence that can be introduced into a host cell to be recombined and inserted into the genome of the host cell, or spontaneously replicated as an episome. The vector may include linear nucleic acids, plasmids, phagemids, cosmids, RNA vectors, virus vectors, and analogs thereof. Examples of the virus vectors may include retroviruses, adenoviruses, and adeno-associated viruses, but are not limited thereto.

As used herein, the term “gene expression” or “expression” of a target protein is understood to refer to transcription of a DNA sequence, translation of an mRNA transcript, and secretion of a fusion protein product or a fragment thereof.

As used herein, the term “host cell” refers to a prokaryotic cell and a eukaryotic cell to which a recombinant expression vector can be introduced. As used herein, the terms “transduced”, “transformed”, and “transfected” refer to the introduction of a nucleic acid (e.g., a vector) into a cell using a technology known in the art.

As used herein, the term “gene expression” or “expression” of a target protein is understood to refer to transcription of a DNA sequence, translation of an mRNA transcript, and secretion of an Fc fusion protein product or an antibody or an antibody fragment thereof.

The useful expression vector may be RcCMV (Invitrogen, Carlsbad) or a variant thereof. The expression vector may include a human cytomegalovirus (CMV) for promoting continuous transcription of a target gene in a mammalian cell and a polyadenylation signal sequence of bovine growth hormone for increasing the stability state of RNA after transcription. In an exemplary embodiment, the expression vector is pAD15, which is a modified form of RcCMV.

The expression vector may be included in an appropriate host cell suitable for the expression and/or secretion of a target protein, by the transduction or transfection of the DNA sequence of an embodiment.

Examples of the appropriate host cell may include, but are not limited to, immortal hybridoma cell, NS/0 myeloma cell, 293 cell, Chinese hamster ovary (CHO) cell, HeLa cell, human amniotic fluid-derived cell (CapT cell), or COS cell.

The modified IL-7 protein and its fusion protein may be produced by a method described in copending U.S. application Ser. No. 15/126,313, of which the entire content is incorporated herein by reference.

Composition and/or Formulation

U.S. application Ser. No. 15/773,273, of which entire content is incorporated herein by reference, discloses a formulation containing a modified IL-7 fusion protein. In some embodiments, a pharmaceutical formulation may comprise (a) a modified IL-7 polypeptide or a modified IL-7 fusion protein; (b) a basal buffer with a concentration of 10 to 50 mM; (c) a sugar with a concentration of 2.5 to 5 w/v %; and (d) a surfactant with a concentration of 0.05 to 6 w/v %. The formulation may further comprise an amino acid, sugar alcohol (e.g., sorbitol, xylitol, maltitol, mannitol, or a mixture thereof). The amount of (a) modified IL-7 polypeptide or a modified IL-7 fusion protein may be contained in the composition in an amount of about 0.5% w/v—about 20% w/v, about 1% w/v/—about 20% w/v, about 1% w/v—about 15% w/v, about 1% w/v—about 10% w/v, about 1% w/v—about 5% w/v, about 2% w/v—about 10% w/v, about 2% w/v—about 5% w/v, or about 2% w/v—about 3% w/v. According to an embodiment, the composition may contain the following components:

| Use | Composition | Amount (1 mL) | |
|---|---|---|---|
| Active pharmaceutical | Modified IL-7 | 25.00 mg (range | |
| ingredient | | 20-30 mg) | |
| Stabilizer | Sucrose | 50 | mg |
| Stabilizer | D-Sorbitol | 15 | mg |
| Buffer | Tri-sodium citrate | 3.68 | mg |
| | dihydrate | | |
| Buffer | Citric acid monohydrate | 1.58 | mg |
| Stabilizer | Polysorbate 80 | 0.5 | mg |

| | | |
|---|---|---|
| Vehicle | Water for Injection | q.s. 1 mL |

A pharmaceutical composition containing the modified IL-7 or an IL-7 fusion protein may be administered to a subject by a direct administration (e.g., locally by an administration via injection, transplantation, or local administration into a tissue region) or system (e.g., parenterally or orally) via an appropriate means. When the composition is administered parenterally by intravenously, subcutaneously, intraocularly, intraperitoneally, intramuscularly, orally, intrarectally, intraorbitally, intracerebrally, intracranially, intraspinally, intraventricularly, intrathecally, intracistenally, intracapsularly, intranasally, or aerosol administration, the composition preferably contains an aqueous or physiologically applicable suspension of body fluids or a part of the solution thereof. As such, the physiologically acceptable carrier or transporter can be added into the composition and delivered to patients, and this does not cause a negative effect on the electrolyte and/or volume balance of patients. Accordingly, the physiologically acceptable carrier or transporter may be a physiological saline.

Additionally, a DNA construct (or a genomic construct) including a nucleic acid including the modified IL-7 or an IL-7 fusion protein may be used as a part of the gene therapy protocol.

For reconstituting or complementing the functions of a desired protein, an expression vector capable of expressing a fusion protein in a particular cell may be administered along with any biologically effective carrier. This may be any formulation or composition that can efficiently deliver a gene encoding a desired protein or an IL-7 fusion protein into a cell in vivo.

For the purpose of gene therapy using a nucleic acid encoding the modified IL-7 or an IL-7 fusion protein, a subject gene may be inserted into a virus vector, a recombinant bacteria plasmid, or a recombinant eukaryotic plasmid. The virus vector may include a recombinant retrovirus, an adenovirus, an adeno-associated virus, and herpes simplex virus-1, etc., transplanted T-cells, or proliferating the in vitro isolated T-cell aggregates.

The nucleic acid encoding the modified IL-7 or an IL-7 fusion protein may be administered in a range from about 0.1 mg to about 200 mg. In an embodiment, the nucleic acid encoding the modified IL-7 or an IL-7 fusion protein may be administered in a range from about 0.6 mg to about 100 mg. In another embodiment, the nucleic acid encoding the modified IL-7 or an IL-7 fusion protein may be administered in a range from about 1.2 mg to about 50 mg.

The modified IL-7 or an IL-7 fusion protein thereof may be administered in a range from about 0.001 mg/kg to about 10 mg/kg. In an embodiment, the modified IL-7 or an IL-7 fusion protein thereof may be administered in a range from about 0.01 mg/kg to about 2 mg/kg. In another embodiment, the modified IL-7 or an IL-7 fusion protein thereof may be administered in a range from about 0.02 mg/kg to about 1 mg/kg. The dose could be determined by a physician, depending on the severity of the symptoms, target disease, side effects, and desired outcome, and the like.

Administration of the modified interleukin-7 protein can be performed by periodic bolus injections, or from an external reservoir (e.g., an intravenous bag) or from the inside (e.g., a biocorrosive implant). It can be by intravenous, subcutaneous or intraperitoneal administration.

### Use of Modified 11-7 or Fusion Protein Thereof and Treatment Regimen

The modified interleukin-7 or a fusion protein thereof may be administered in combination with other drugs or physiologically active substances having a preventive or therapeutic effect on the disease to be prevented or treated, or it can be formulated into a combination formulation drug together with other drugs. For example, it can be used in combination with one or more immunostimulating agents. Immunostimulating agent may be exemplified by hematopoietic cell growth factors, cytokines, antigens, and adjuvants. Hematopoietic cell growth factors may be a Stem Cell Factor (SCF), G-CSF, GM-CSF, or Flt-3 ligand. Cytokines may be gamma interferon, IL-2, IL-15, IL-21, IL-12, RANTES or B7-1.

When the modified IL-7 or its fusion protein is used in combination with other drugs or physiologically active substances, the route of administration, timing of administration, and dosage may be determined depending on the type of disease, disease state of the patient, the purpose of treatment or prevention, and the kind of other drugs or physiologically active substances used in combination.

Use of the modified IL-7 or its fusion protein for use in the treatment of a proliferative disease comprising administering a compound of formula (I) or a pharmaceutically acceptable salt thereof in a therapeutically effective dosage of about 720 μg/kg or above, about 960 μg/kg or above, about 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein for use in the treatment of a proliferative disease comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to a subject in need thereof.

Use of the modified IL-7 or its fusion protein for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 960 μg/kg or above, about 1,200 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

Use of the modified IL-7 or its fusion protein for use in increasing a lymphocyte count or lymphocyte production in a subject in need thereof comprising administering a compound of formula (I) or a pharmaceutically acceptable salt thereof in a therapeutically effective dosage of about 720 μg/kg or above, about 840 μg/kg or above, about 960 μg/kg or above, about 1,200 μg/kg or above, about 1,440 μg/kg or above, about 1,700 μg/kg or above, or about 2,000 μg/kg of the modified IL-7 or its fusion protein twice or more times at an interval of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks, or at an interval of 10 days, 20 days, 30 days, 40 days, 50 days, 60 days, 70 days, 80 days, 90 days, or 100 days, to the subject.

In an embodiment, the proliferative disease for the above uses is cancer or tumor, which may be, but not limited to, a solid tumor, a cancer of lymphatic system, or leukemia.

The solid tumor may be synovial sarcoma, infiltrating duct carcinoma, rectal cancer, colon cancer, ovary cancer, ascending colon cancer, anal cancer, invasive ductal carcinoma, adenocarcinoma, rectal cancer with paraaortic in metastasis, neuroendocrine carcinoma (cervix), sigmoid colon cancer, or glioblastoma.

In an embodiment, the disease for the above uses is infectious disease. The infectious disease may be a viral infection, and the virus may be selected from the group consisting of influenza virus, human papilloma virus (HPV), cytomegalovirus (CMV), herpes simplex type 1 virus (HSV-1), herpes simplex type 2 (HSV-2), human immunodeficiency virus (HIV), hepatitis C virus (HCV), hepatitis B virus (HBV), norovirus, rotavirus, rabies virus, Zika virus, rubella virus, West Nile fever virus, Dengue virus, eterovirus (such as coxackieiruses and echoviruses), SARS-CoV, SARS-CoV2, Epstein-Barr virus, and the like.

In an embodiment, the subject could have previously received or concurrently receives one or more of cancer treatments including surgery, radiation, and chemotherapy.

The (i) modified interleukin-7 or the (ii) interleukin-7 fusion protein, or a pharmaceutical composition containing them may be administered parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

In some aspects, the subject may have a lymphocyte count of 1000 lymphocyte cells or less/μl of blood as determined according to Common Terminology Criteria for Adverse Events (CTCAE) version 4.0. The lymphocyte may be T-cell. The T-cell may include CD4+ and/or CD8+ T-cell. For the measurement of lymphocytes in blood sample, whole blood or serum may be used. Therefore, the term “blood” used in relation with the lymphocyte count includes whole blood and/or serum.

In another aspect, the subject has a lymphocyte count of 800 lymphocyte cells or less/μl of blood, a lymphocyte count of about 500 lymphocyte cells or less/μl of blood, or a lymphocyte count of about 200 lymphocyte cells or less/μl of blood.

In an aspect, a number of tumor infiltrating lymphocytes (TILs) in the tumor is increased after the administration compared to a number of TILs in a tumor after administration of the (i) modified interleukin-7 or the (ii) interleukin-7 fusion protein. The TILs may be CD4⁺ TILs and/or CD8⁺ TILs.

The number of TILs may be increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration.

### EXAMPLES

Hereinafter, the present disclosure is explained in detail by Examples. The following Examples are intended to further illustrate the present invention without limiting its scope.

### Example 1. IL-7 Polypeptide Structure and Predicted Structure Analysis

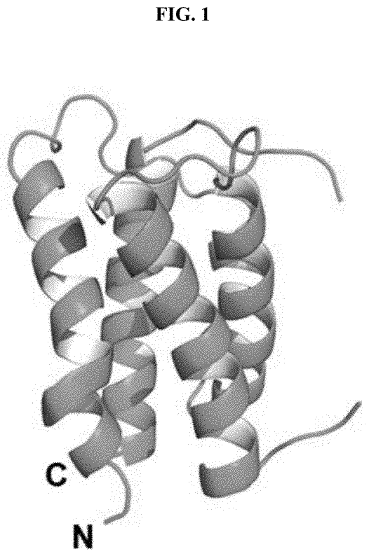

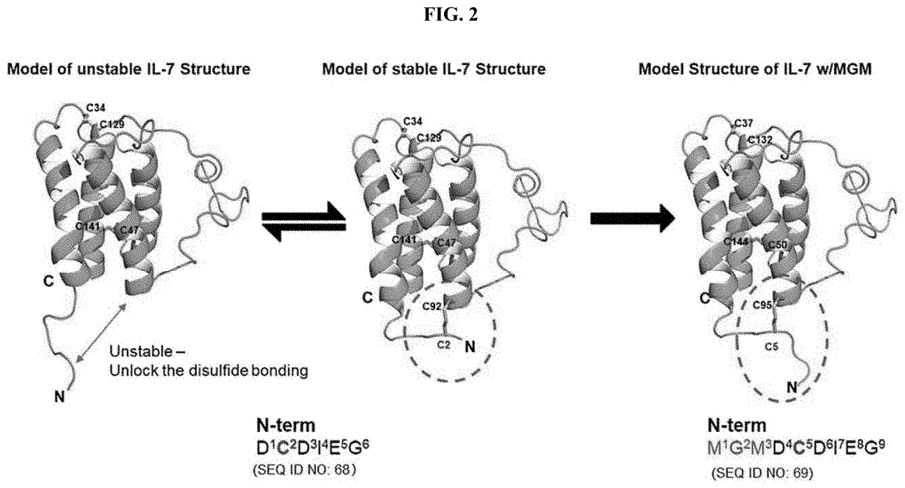

IL-7 includes five (5) alpha-helix bundles and maintains structural stability by forming disulfide bonding through six (6) cysteines. In addition, the region from amino acids at positions 1 to 6 from the N-terminal are very flexible and are not seen in the X-ray crystal structure, as illustrated in FIG. 1 (see, also Structure, 2009, McElroy et al.) It is suggested that amino residues preceding the alpha-helical structure play an important role in the formation of the helix (Structural biology, 1995, Munoz et al.). And since interleukin-7 contains five alpha-helix bundles, the inventors postulated that an unstable formation of the first alpha helix of IL-7 could cause a low or difficult formation of disulfide bonding between the first cysteine (C2) and the fourth cysteine (C92). The inventors further postulated that such an unstable first disulfide formation could result in an IL-7 structurally unstable and reduce an expression level. The inventors conceived that an addition of an oligopeptide to the N-terminal of IL-7 could change the secondary structure of the IL-7 and form a structurally stable IL-7, as illustrated in FIG. 2.

### Example 2. Preparation of Modified IL-7 Polypeptide

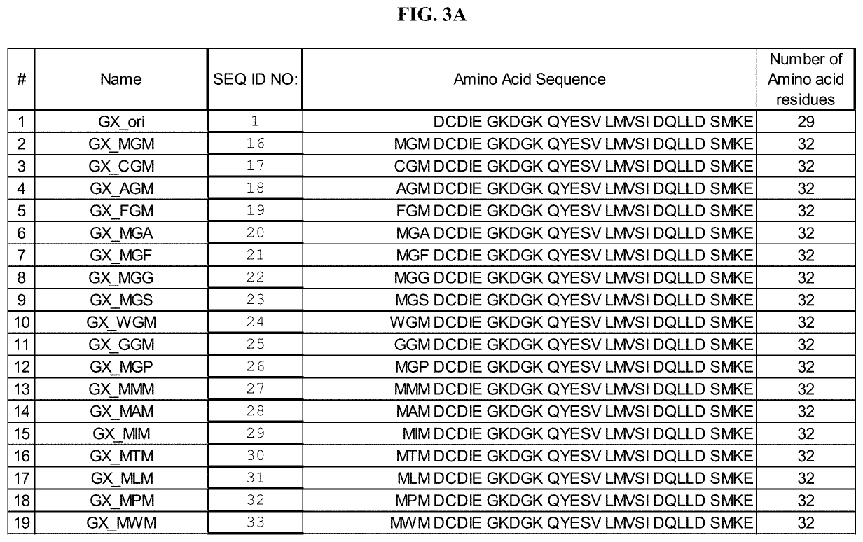

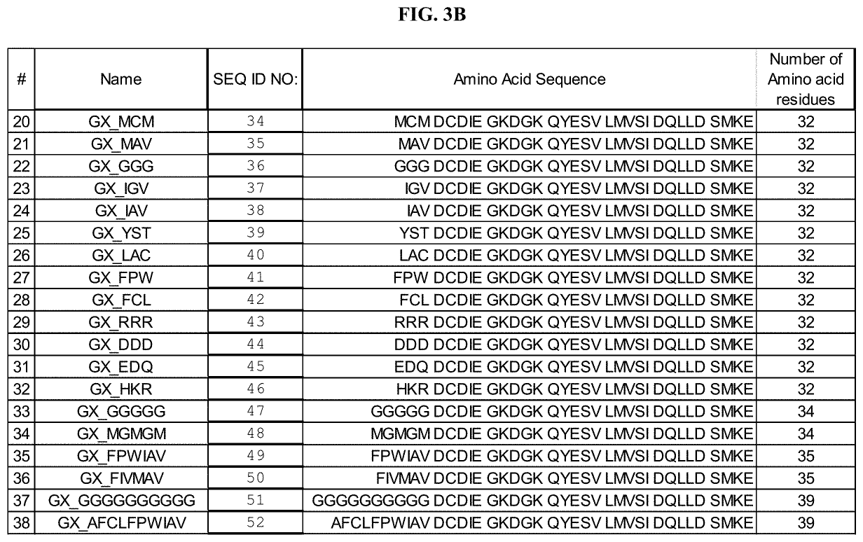

Human IL-7 comprises 29 amino acid residues (DCDIEG KDGKQYESVLMVSIDQLLDSMKE) (SEQ ID NO: 1). Wild-type human IL-7 and modified IL-7 polypeptides as shown in FIG. 3A and FIG. 3B are manufactured by oligomer synthesis. The purity of the synthesized modified IL-7 polypeptides were tested by Revers phase HPLC. All of the modified IL-7 showed 95% or higher.

### Example 3. Circular Dichroism of the Modified IL-7 Polypeptide

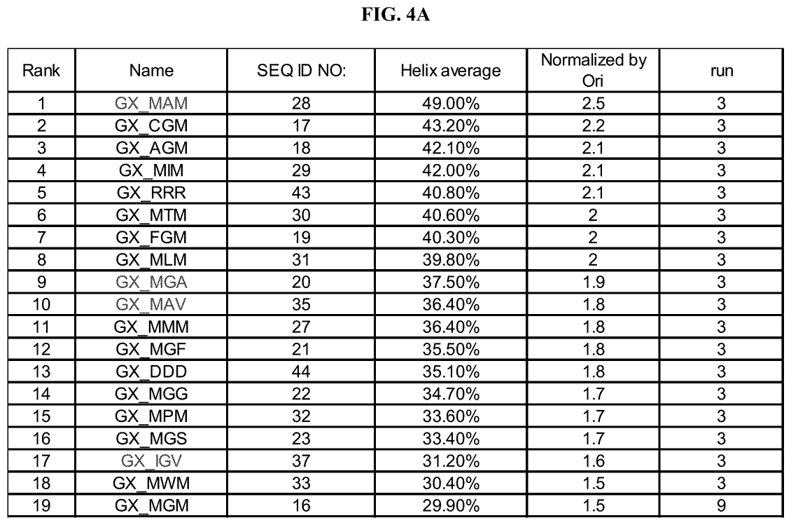

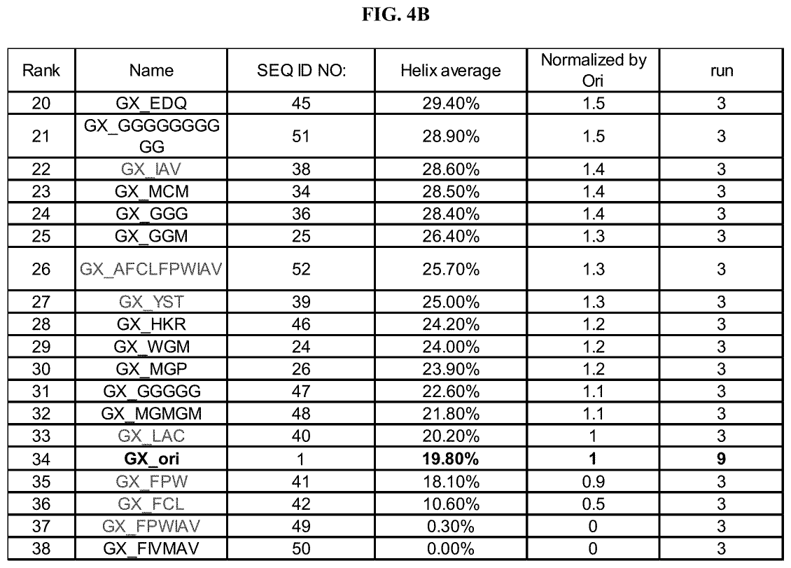

The modified IL-7 polypeptides and the wild-type IL-7, shown in FIG. 3A and FIG. 3B were dissolved in 50% (v/v) acetonitrile buffer to a final concentration 1 mg/ml. Circular dichroism (CD) were analyzed by measuring Far UV at the 190 nm-250 nm frequencies using JASCO J-715, and the measured CD values were used to obtain alpha-helix formation index. CD were carried out three times or more and the alpha-helix formation index was obtained as an average. The results are shown in FIGS. 4A and 4B.

Among the tested 38 modified IL-7 polypeptides, GX-MAM (SEQ ID NO: 28, IL-7 having MAM at the N-terminal) showed the highest formation index (49.0%) and GX-FIVMAV (SEQ ID NO: 50, IL-7 having FIVMAV at the N-terminal). GX-MAM showed about 1.5 times greater of alpha-helix formation than the wild-type IL-7 (SEQ ID NO: 1).

The helix formation index (helix average) of GX_AFCLFPWIAV (SEQ ID 0: 52)(25.7%) and GX_GGGGGGGGGG (SEQ ID NO: 51)(28.9%) were similar to GX_MGM (SEQ ID NO: 16)(29.9%), and GX_GGGGG (SEQ ID NO: 47)(22.6%) and GX_MGMGM (SEQ ID NO: 48)(21.8%) were similar to GX_ori (SEQ ID NO: 1)(19.8%), but GX_FIVMAV (SEQ ID NO: 50)(0%), GXFPWIAV (SEQ ID NO: 49)(0.3%) had lower alpha helix formation than GX_ori (SEQ ID NO: 1). No meaningful differences in alpha helix formations according to the length of the oligopeptide A were observed. It was observed that the alpha helix formation of modified IL-7 according to the embodiments could be dependent on the sequence of oligopeptide A.

In addition, it can be seen that the alpha helix formation of the charged amino acid oligopeptides (GX_RRR (SEQ ID NO: 43), GX_DDD (SEQ ID NO: 44), GX_EDQ (SEQ ID NO: 45), GX_HKR) (SEQ ID NO: 46) is greater than that of GX_ori. This is because the oligopeptide having a pI (isoelectric point) of 4.0, exhibits a positive charge in a pH 3.0 buffer and a negative charge in a pH 7.0 buffer.

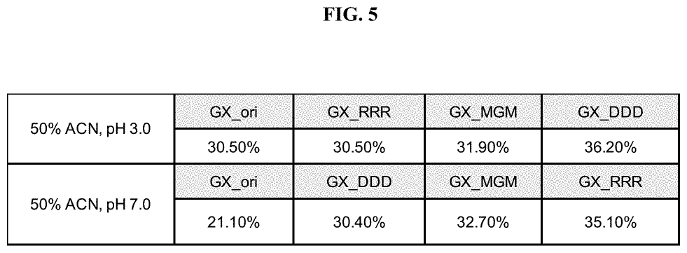

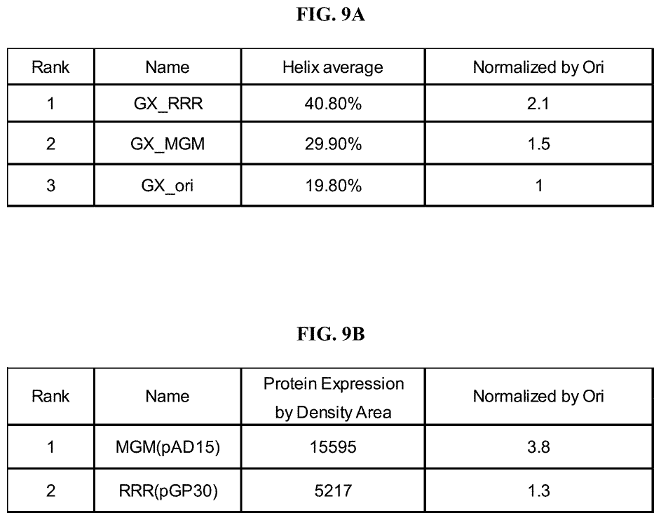

This explains that GX_DDD (SEQ ID NO: 44) shows a higher helix formation than GX_RRR (SEQ ID NO: 43) at pH 3.0, which the inventors speculate without limitation to other possible explanations is attributed to the combination of surrounding positive charge and aspartic acid (D, negative amino acid) in the vicinity. In pH 7.0, GX_FFF (SEQ ID NO: 43) shows a higher helix formation than GX_DDD (SEQ ID NO: 44), possibly through the combination of surrounding negative charge and arginine (positive amino acid). When a fusion protein of GX_RRR was expressed, the expression level was lower than the fusion protein of GX_MGM. Thus, for the modified IL-7 polypeptides having charged amino acid residues as “A,” a higher helix formation of a modified IL-7, which is influenced by environmental electric charges, is not always translated into a higher expression level of a fusion protein. See, FIGS. 5, 8, 9A, and 9B.

### Example 4: Preparation and Expression of Modified Interleukin-7 Fusion Proteins

To examine the relationship between alpha-helix formation (measured by CD analysis) of a modified IL-7 and an expression level of a fusion protein containing the modified IL-7, polynucleotides encoding various fusion proteins (A-IL-7-hyFc) were constructed.

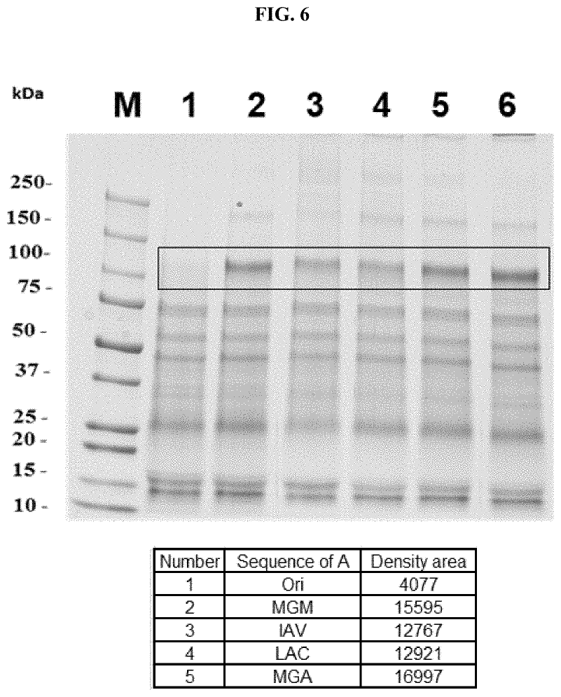

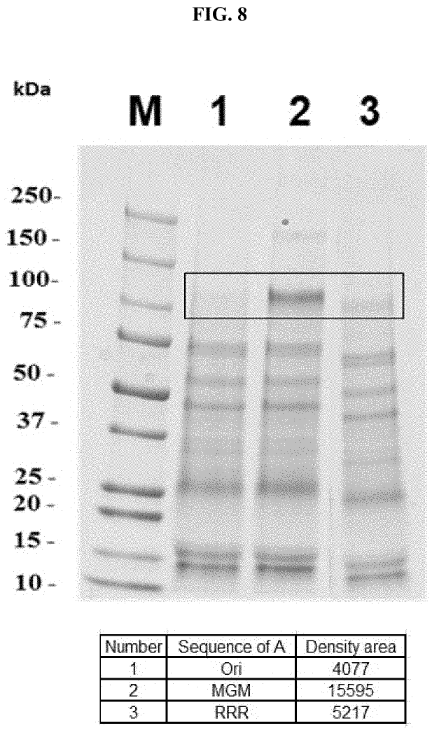

The hyFc of the modified IL-7 fusion proteins, as employed in the studies, is a modified Fc region of SEQ ID NO: 9, as described in U.S. Pat. No. 7,867,491, the entire content of which is incorporated herein by reference. The expression vectors comprising the modified IL-7—hyFc fusion protein—encoding polynucleotide were used to express the proteins in EXPICHO™ transient production system. It was confirmed that all of the fusion proteins were expressed as demonstrated in SDS-PAGE results of FIGS. 6 and 8, where the fusion proteins are detected at about 100 kDa under non-educing conditions. Exemplary fusion proteins obtained in Example 4 comprise the amino acid sequence of SEQ ID NO: 54 to 59. The amino acid sequence of SEQ ID NO: 54-59 could be obtained by expressing the polynucleotide of SEQ ID NOS: 61-66 under conditions allowing expression of the fusion protein.

### Example 5: Comparison of Alpha-Helix Formation of Modified IL-7 and Expression of Fusion Proteins

Among modified IL-7 peptides, GX_MAM (SEQ ID NO: 28) (49.0%) and GX_MGA (SEQ ID NO: 20) (37.5%) showed a higher alpha-helix formation index than GX_MGM (SEQ ID NO: 16)(29.9%), and GX_IAV (SEQ ID NO: 38)(28.6%) and GX_LAC (SEQ ID NO: 40) (20.2%) were smaller than GX_MGM (SEQ ID NO: 16) and GX_Ori (SEQ ID NO: 1)(19.8%). See results in FIGS. 4A and 4B.

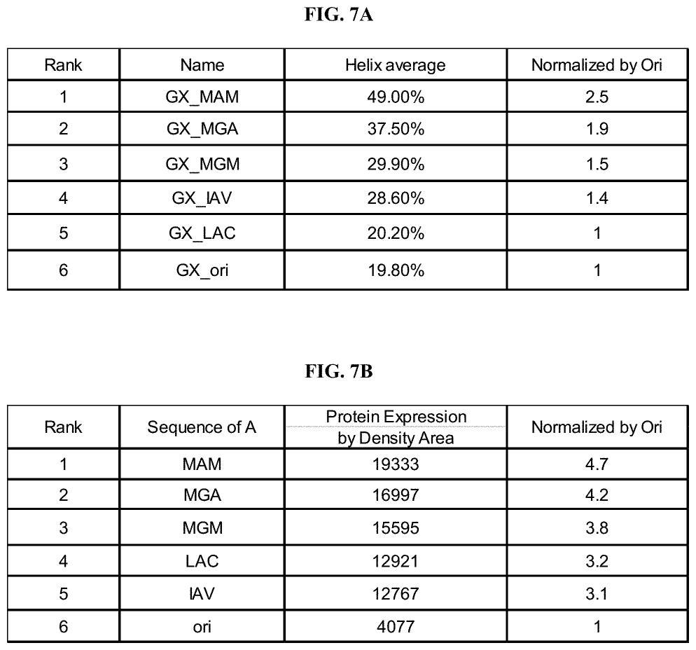

With regard to expressions of the fusion proteins employing the EXPICHO™ transient production system, the expression levels were in this order of GX_MAM (SEQ ID NO: 28) (19333), GX_MGA (SEQ ID NO: 20) (16997), GX_MGM (SEQ ID NO: 16) (15595), GX_LAC (SEQ ID NO: 40) (12921), GX_IAV (SEQ ID NO: 38) (12767), GX_Ori (SEQ ID NO: 1) (4077), as shown in FIGS. 6, 7A, and 7B. In FIGS. 6, 7A, and 7B, the expression levels were shown as a density area obtained from Image J.

### Experimental Example 1: Study of Pharmacokinetic Profiles of Modified IL-7 and IL-7 Fusion Proteins

The pharmacokinetic profiles (PK) are confirmed by comparing half-life and area under the curve (AUC) of a fusion protein comprising the sequence of SEQ ID NO: 54, 55, 56, 57, 58, or 59.

Male Sprague Dawley (SD) rats (5 rats/group) are subcutaneously administered with each of the fusion proteins in an amount of 0.2 mg/kg, respectively. Blood samples are collected before the administration and at 4, 8, 12, 24, 48, 72, 96, 120, 144, and 168 hours after the administration, and stored at room temperature for 30 minutes to aggregate the blood samples. The aggregated blood samples are centrifuged at 3,000 rpm for 10 minutes and the blood serum of each sample is obtained and stored in a deep-freezer.

The samples are analyzed by a test method designed to specifically detect the intact form of the fusion protein in which no cleavage occurred. This method detects a target protein using the secondary antibody (Southern Biotech, Cat #9190-05), to which horseradish peroxidase (HRP) coupled to the human immunoglobulin G4 (IgG4) of mouse origin is conjugated, after loading a sample containing the fusion protein into a plate coated with a capture antibody of mouse origin (R&D, Cat #MAB207), which is coupled to the human IL-7. The samples are quantitated by a 10-fold dilution with 1×PBS containing 10% skim milk to be analyzed in a linear position of a standard curve. The results are obtained in terms of the protein amount remaining in the sample per each time point and the drug concentration area under the curve.

### Experimental Example 2. Study of Immunogenicity of Modified IL-7 and IL-7 Fusion Proteins

Anti-drug antibody (ADA) producing ability of fusion proteins comprising the modified IL-7 according to embodiments described herein is determined. The blood samples obtained in the same manner as described in Experimental Example 1 are loaded into the plate, which is coated with the fusion proteins (e.g., fusion proteins comprising the sequence of SEQ ID NO: 54, 55, 56, 57, 58, or 59) in an amount of 0.2 ug/well. Then, the samples are analyzed using the test method designed to detect the ADA in rats using the rat immunoglobulin antibody (Southern Biotech, Cat #1031-05), to which HRP is conjugated.

### Experimental Example 3. Study of Pharmacodynamic Profile of IL-7 Fusion Proteins

A change in the number of white blood cells (WBC) by administering the fusion proteins according to embodiments of the instant disclosure is determined. Male SD rats (5 rats/group) are subcutaneously administered with each of the fusion proteins (e.g., fusion proteins comprising the sequence of SEQ ID NO: 54, 55, 56, 57, 58, or 59) in an amount of 0.2 mg/kg. Then, blood samples are collected from the rats before the administration and on the 1^{st}, the 2^{nd}, and the 3^{rd} week after the administration. To prevent the aggregation of the blood samples, the samples are obtained in EDTA-treated tubes, mixed for 5 minutes and stabilized, and the number of WBC is analyzed by complete blood count (CBC) analysis.

### Experimental Example 4. In Vitro Activity of IL-7 Fusion Proteins

2E8 cells (ATCC, TIB-239), murine immature B lymphocytes, are seeded into a 96-well plate (1×10⁵ cells/50 μL/well), and each of the fusion proteins according to embodiments (e.g., protein comprising the sequence of SEQ ID NO: 54, 55, 56, 57, 58, or 59) is stepwise diluted at a concentration of 750 μM to 2.93 μM and treated on the wells. The cells are cultured in an incubator (37° C., 5% CO₂) for 70 hours, treated with MTS at a concentration of 20 μL/well, and cultured again in the incubator (37° C., 5% CO₂) for 4 hours. Then, the absorbance is measured at 490 nm. WHO international standard human IL-7 (NIBSC code: 90/530, 100,000 unit) is used as control group. The calibration curve according to the concentration of the fusion protein treatment is created (4-parameter fit).

### Experimental Example 5. Study of Prevention and Treatment Effect in an Influenza-Infected Animal Model

The mouse model with a lethal dose of influenza is prepared by anesthetizing followed by the administration with 3LD₅₀ H5N2 virus (A/Aquatic bird/Korea/W81/2005) through nasal cavity. Generally, virus-infected mice begin to lose their body weight from 2-3 days and die from a week and thereafter. The thus-prepared disease model is administered with one of the fusion proteins according to the embodiments (e.g., protein comprising the sequence of SEQ ID NO: 54, 55, 56, 57, 58, or 59), and the wild-type IL-7-mFc (mouse Fc) fusion protein is used as a control group.

As G-CSF can inhibit the early stage influenza infection and proliferate neutrophils thereby promoting immune response, G-CSF-hyFc is used as comparative group.

Six mice are used per each experimental group, and IL-7-mFc (IL-7-mouse Fc), one of the fusion proteins, or G-CSF-hyFc is administered through nasal cavity 14 days before the infection with a lethal dose of influenza. Then, 3LD₅₀ H5N2 virus is administered and the body weight and survival rate are observed for 20 days.

### Experimental Example 6. Study of Treatment Effect in TC-1 Cancer Animal Model

An animal model with endometrial cancer is prepared by administering intraperitoneally 3 mg of Depo-Provera to adjust the period of their menstruation. In 4 days, the mice are administered with nonoxynol-9 (N9, USP, Cat. No. 1467950) into the vagina to stimulate the vagina tissue growth, and the remaining N9 is removed by washing with PBS. Then, for the transplant of the cancer cells into the uterus, 1×10⁵ TC-1 cell (Dr. Jae-Tae LEE, School of Medicine, Kyungpook National University) are administered, and then 1 day thereafter, 1 ug of the fusion protein (e.g., protein comprising the sequence of SEQ ID NO: 54, 55, 56, 57, 58, or 59) is administered into the uterine cervix. Distilled water is administered as a control.

28 days after the administration of the fusion protein, the TC-1 tumor cells are observed.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

We claim:

1. A modified interleukin-7 having the following structure:
A-IL-7
wherein A is an oligopeptide consisting of 1 to 10 amino acid residues, and
IL-7 is an interleukin-7 or a polypeptide capable of binding to interleukin-7 receptor, said polypeptide having an amino acid sequence identify of 85% or more to SEQ ID NO: 1,
with proviso that the A excludes an oligopeptide consisting of methionine and/or glycine.

2. The modified interleukin-7 of claim 1, wherein the A is an oligopeptide selected from the group consisting of (a)-(c):
(a) an oligopeptide comprising methionine and/or glycine, and one or more hydrophobic amino acid residues selected from the group consisting of isoleucine, valine, leucine, phenylalanine, cysteine, alanine, tryptophan, and proline,
(b) an oligopeptide consisting of a hydrophobic amino acid residue selected from the group consisting of isoleucine, valine, leucine, phenylalanine, cysteine, alanine, tryptophan, and proline, and
(c) an oligopeptide consisting of a hydrophilic amino acid residue selected from the group consisting of asparagine, aspartic acid, glutamine, glutamic acid, lysine, arginine, histidine, tyrosine, threonine, and serine.

3. The modified interleukin-7 of claim 1, wherein the oligopeptide A comprises
(i) methionine and alanine;
(ii) methionine and cysteine;
(iii) methionine and valine;
(iv) methionine and phenylalanine;
(v) methionine and leucine;
(vi) methionine and serine;
(vii) methionine and proline;
(viii) methionine and isoleucine;
(ix) methionine and tryptophan;
(x) methionine and threonine;
(xi) methionine, glycine, and alanine;
(xii) methionine, glycine, and cysteine;
(xiii) methionine, glycine, and valine;
(xiv) methionine, glycine, and phenylalanine;
(xv) methionine, glycine, and leucine;
(xvi) methionine, glycine, and serine;
(xvii) methionine, glycine, and isoleucine;
(xviii) methionine, glycine, and tryptophan;
(xix) methionine, glycine, and threonine; or
(xx) methionine, glycine, and proline.

4. The modified interleukin-7 of claim 1, wherein the A is any one selected from the group consisting of alanine, cysteine, methionine-alanine, methionine-alanine-methionine, methionine-alanine-methionine-methionine-alanine-methionine, methionine-alanine-methionine-methionine-alanine-methionine-methionine-alanine-methionine, cysteine-glycine-methionine, cysteine-glycine-methionine-methionine-alanine-methionine, cysteine-glycine-methionine-cysteine-glycine-methionine, alanine-glycine-methionine, cysteine-glycine-methionine-alanine-glycine-methionine, methionine-isoleucine-methionine, methionine-alanine-methionine-isoleucine-methionine-methionine-isoleucine-methionine-methionine-methionine, cysteine-glycine-methionine-alanine-glycine-methionine, methinone-alanine-methionine-alanine-methionine-glycine-methionine-methionine-glycine-glycine, arginine-arginine-arginine, methionine-threonine-methionine, phenylalanine-glycine-methionine, methionine-leucine-methionine, methionine-glycine-alanine, methionine-alanine-valine, methionine-glycine-phenylalanine, aspartic acid-aspartic acid-aspartic acid, methionine-proline-methionine, methionine-glycine-serine, isoleucine-glycine-valine, methionine-tryptophan-methionine, glutamic acid-aspartic acid-glutamine, isoleucine-alanine-valine, methionine-cysteine-methionine, alanine-phenylalanine-cysteine-leucine-phenylanaline-proline-tryptophan-isoleucine-alanine-valine, tyrosine-serine-threonine, histidine-lysine-arginine, tryptophan-glycine-methionine, methionine-glycine-proline, leucine-alanine-cysteine, phenylalanine-proline-tryptophan, phenylalanine-cysteine-leucine, phenylalanine-proline-tryptophan-isoleucine-alanine-valine, and phenylalanine-isoleucine-valine-methionine-alanine-valine.

5. The modified interleukin-7 of claim 1, wherein the A is any one selected from the group consisting of methionine-glycine-alanine, methionine-alanine-methionine, arginine-arginine-arginine, cysteine-glycine-methionine, alanine-glycine-methionine, methionine-isoleucine-methionine, methionine-threonine-methionine, phenylalanine-glycine-methionine, methionine-leucine-methionine, methionine-alanine-valine, methionine-glycine-phenylalanine, aspartic acid-aspartic acid-aspartic acid, methionine-proline-methionine, and methionine-glycine-serine.

6. The modified interleukin-7 of claim 1, wherein the A is any one selected from the group consisting of leucine-alanine-cysteine, isoleucine-alanine-valine, isoleucine-glycine-valine, methionine-tryptophan-methionine, glutamic acid-aspartic acid-glutamine, methionine-cysteine-methionine, tyrosine-serine-threonine, histidine-lysine-arginine, tryptophan-glycine-methionine, and methionine-glycine-proline.

7. The modified interleukin-7 of claim 1, wherein the IL-7 has an amino acid sequence selected from the group consisting of SEQ ID NOS: 1 to 6.

8. The modified interleukin-7 of claim 1, wherein A is linked to the N-terminal of the IL-7.

9. The modified interleukine-7 of claim 1, which comprises any one of the sequence selected from the group consisting of SEQ ID NOS: 17-21, 23, 24, 26, 28-35, 37-46, 49, 50, and 52.

10. A fusion protein comprising
(a) a first domain comprising an interleukin-7, wherein the interleukin-7 comprises the amino acid sequence of SEQ ID NO: 1, 2, 3, 4, 5, or 6, or a polypeptide capable of binding to interleukin-7 receptor and having an amino acid sequence identify of 85% or more to SEQ ID NO: 1;
(b) a second domain comprising an oligopeptide having 1 to 10 amino acid residues, with proviso that the second domain excludes an oligopeptide consisting of methionine and/or glycine; and
(c) a third domain which prolongs the half-life of the IL-7 fusion protein.

11. The fusion protein of claim 10, wherein the second domain is an oligopeptide selected from the group consisting of (a)-(c):
(a) an oligopeptide comprising methionine and/or glycine, and one or more hydrophobic amino acid residues selected from the group consisting of isoleucine, valine, leucine, phenylalanine, cysteine, alanine, tryptophan, and proline,
(b) an oligopeptide consisting of a hydrophobic amino acid residue selected from the group consisting of isoleucine, valine, leucine, phenylalanine, cysteine, alanine, tryptophan, and proline, and
(c) an oligopeptide consisting of a hydrophilic amino acid residue selected from the group consisting of asparagine, aspartic acid, glutamine, glutamic acid, lysine, arginine, histidine, tyrosine, threonine and serine.

12. The fusion protein of claim 10, wherein the second domain is an oligopeptide comprising:
(i) methionine and alanine;
(ii) methionine and cysteine;
(iii) methionine and valine;
(iv) methionine and phenylalanine;
(v) methionine and leucine;
(vi) methionine and serine;
(vii) methionine and proline;
(viii) methionine and isoleucine;
(ix) methionine and tryptophan;
(x) methionine and threonine;
(xi) methionine, glycine, and alanine;
(xii) methionine, glycine, and cysteine;
(xiii) methionine, glycine, and valine;
(xiv) methionine, glycine, and phenylalanine;
(xv) methionine, glycine, and leucine;
(xvi) methionine, glycine, and serine;
(xvii) methionine, glycine, and isoleucine;
(xviii) methionine, glycine, and tryptophan;
(xix) methionine, glycine, and threonine; or
(xx) methionine, glycine, and proline.

13. The fusion protein of claim 10, wherein the second domain is any one selected from the group consisting of alanine, cysteine, methionine-alanine, methionine-alanine-methionine, methionine-alanine-methionine-methionine-alanine-methionine, methionine-alanine-methionine-methionine-alanine-methionine-methionine-alanine-methionine, cysteine-glycine-methionine, cysteine-glycine-methionine-methionine-alanine-methionine, cysteine-glycine-methionine-cysteine-glycine-methionine, alanine-glycine-methionine, cysteine-glycine-methionine-alanine-glycine-methionine, methionine-isoleucine-methionine, methionine-alanine-methionine-isoleucine-methionine-methionine-isoleucine-methionine-methionine-methionine, cysteine-glycine-methionine-alanine-glycine-methionine, methinone-alanine-methionine-alanine-methionine-glycine-methionine-methionine-glycine-glycine, arginine-arginine-arginine, methionine-threonine-methionine, phenylalanine-glycine-methionine, methionine-leucine-methionine, methionine-glycine-alanine, methionine-alanine-valine, methionine-glycine-phenylalanine, aspartic acid-aspartic acid-aspartic acid, methionine-proline-methionine, methionine-glycine-serine, isoleucine-glycine-valine, methionine-tryptophan-methionine, glutamic acid-aspartic acid-glutamine, isoleucine-alanine-valine, methionine-cysteine-methionine, alanine-phenylalanine-cysteine-leucine-phenylanaline-proline-tryptophan-isoleucine-alanine-valine, tyrosine-serine-threonine, histidine-lysine-arginine, tryptophan-glycine-methionine, methionine-glycine-proline, leucine-alanine-cysteine, phenylalanine-proline-tryptophan, phenylalanine-cysteine-leucine, phenylalanine-proline-tryptophan-isoleucine-alanine-valine, phenylalanine-isoleucine-valine-methionine-alanine-valine.

14. The fusion protein of claim 10, wherein the second domain is any one selected from the group consisting of methionine-glycine-alanine, methionine-alanine-methionine, arginine-arginine-arginine, cysteine-glycine-methionine, alanine-glycine-methionine, methionine-isoleucine-methionine, methionine-threonine-methionine, phenylalanine-glycine-methionine, methionine-leucine-methionine, methionine-alanine-valine, methionine-glycine-phenylalanine, aspartic acid-aspartic acid-aspartic acid, methionine-proline-methionine, and methionine-glycine-serine.

15. The fusion protein of claim 10, wherein the second domain is any one selected from the group consisting of leucine-alanine-cysteine, isoleucine-alanine-valine, isoleucine-glycine-valine, methionine-tryptophan-methionine, glutamic acid-aspartic acid-glutamine, methionine-cysteine-methionine, tyrosine-serine-threonine, histidine-lysine-arginine, tryptophan-glycine-methionine, and methionine-glycine-proline.

16. The fusion protein of claim 10, wherein the third domain (c) is linked to the N-terminal or C-terminal of the first domain (a) or the second domain (b).

17. The fusion protein of claim 10, wherein the third domain (c) is any one selected from the group consisting of an Fc region of immunoglobulin or a part thereof, albumin, an albumin-binding polypeptide, Pro/Ala/Ser (PAS), a C-terminal peptide (CTP) of the 13 subunit of human chorionic gonadotropin, polyethylene glycol (PEG), long unstructured hydrophilic sequences of amino acids (XTEN), hydroxyethyl starch (HES), an albumin-binding small molecule, and a combination thereof. In an embodiment, the third domain comprises an Fc region of a modified immunoglobulin. In an embodiment, the modified immunoglobulin is selected from the group consisting of IgG1, IgG2, IgG3, IgG4, IgA1, IgA2, IgD, IgE and a combination thereof.

18. The fusion protein of claim 10, wherein the third domain (c) is an Fc region of the following Formula (II):
N′—(Z1)p-Y—Z2-Z3-Z4-C′ Formula (II)
wherein N′ is the N-terminal of a polypeptide and C′ is the C-terminal of a polypeptide;
p is an integer of 0 or 1;
Z1 is an amino acid sequence having 5 to 9 consecutive amino acid residues starting from the amino acid residue at position 98 of the positions from 90 to 98 of SEQ ID NO: 7;
Y is an amino acid sequence having 5 to 64 consecutive amino acid residues starting from the amino acid residue at position 162 of the positions from 99 to 162 of SEQ ID NO: 7;
Z2 is an amino acid sequence having 4 to 37 consecutive amino acid residues starting from the amino acid residue at position 163 of the positions from 163 to 199 of SEQ ID NO: 7;
Z3 is an amino acid sequence having 71 to 106 consecutive amino acid residues starting from the amino acid residue at position 220 of the positions from 115 to 220 of SEQ ID NO: 8; and
Z4 is an amino acid sequence having 80 to 107 consecutive amino acid residues starting from the amino acid residue at position 221 of the positions from 221 to 327 of SEQ ID NO: 8.

19. The fusion protein of claim 18, wherein the fusion protein comprises any one of the amino acid sequence selected from the group consisting of SEQ ID NO: 54, 55, 57, 58, and 59.
